(19) United States
(12) Patent Application Publication
(10) Pub. No.: US 2004/0135970 A1
(43) Pub. Date: Jul. 15, 2004

(54) Ophthalmic refractometer and method of operating an ophthalmic refractometer

(76) Inventor: Gert Koest, Hannover (DE)

Correspondence Address: FLYNN THIEL BOUTELL & TANIS, P.C., 2026 RAMBLING ROAD, KALAMAZOO, MI, 49008-1699, US

(21) Appl. No.: 10/728,144

(22) Filed: Dec. 4, 2003

(30) Foreign Application Priority Data
Jan. 9, 2003 (DE) 103 00 322.3

(51) Int. Cl. A61B 3/10

(52) U.S. Cl. 351/212

(57) ABSTRACT

This invention relates to an ophthalmic refractometer (13) for objective determination of the refractive power of an eye (A) comprising an optometer system for imaging a test mark on the retina of the eye (A) and comprising an observation system (15) for observation of the test mark (18) imaged on the retina, whereby the optometer system and the observation system can be adjusted in mutual synchronization in relation to a reference setting, and a refractive power parameter of the eye (A) can be determined from the difference between the setting at which the test mark (18) is imaged on the retina at least partially with sharp contours and the reference setting. A digital recording device (42) and a digital image processing unit are provided on the observation system, whereby digital image data of the test mark (18) imaged on the retina can be recorded using the recording device (42) and the resulting image data can be analyzed in the image processing unit by digital image processing to determine the refractive power parameter.

This invention relates to an ophthalmic refractometer and a method of operating an ophthalmic refractometer according to the preamble of the independent patent claims.

Such ophthalmic refractometers are known from the state of the art and are used for objective determination of the refractive power of the eye. Ophthalmic refractometry forms an important basis for early detection of eye defects and for the production of eyeglasses.

The human eye functions by detecting and focusing rays of light. The rays of light are focused in their passage through the cornea, the aqueous humor, the lens and the hyaloid body, also known as the vitreous humor. After passing through these components of the eye, the focal point of the light is ideally situated exactly on the retina, so that a sharply contoured image of the environment is formed on the retina. Normal vision (emmetropia) or the absence of errors of refraction is thus characterized by the focal point of the light entering the eye from an infinite distance and striking and being focused on the retina.

Because of errors of refraction in the eye, many people suffer from poor vision, which is characterized in that the focal point of the light does not strike the retina but instead strikes a point just in front of the retina (nearsightedness, also known as myopia) or a point just behind it (farsightedness, also known as hypermetropia). Such a first-order defect in refractive power can be corrected with a spherical lens, so that the combination of the spherical lens with the lens system of the eye results in the light being focused on the retina.

Superimposed on this first-order refractive power defect may also be a second-order refractive power defect or a so-called astigmatism. Astigmatism occurs when the eye has different errors of refraction at different meridians. Such a second-order refractive power defect can be compensated by the additional combination of a cylindrical lens with the spherical lens. In producing eyeglasses suitable for compensating for refractive power defects of the first and second orders, it is therefore conventional to give a set of parameters, including three parameters which unambiguously describe the refractive power defects of the first and second order. The first refractive power parameter D1 indicates the refractive power of the eye in the direction of the first main axis. The second refractive power parameter D2 indicates the refractive power of the eye at the second main axis. The refractive power parameters D1 and D2 are usually given in diopters. The third refractive power parameter α indicates the angle of the main axes in relation to the vertical or the horizontal and ultimately corresponds to the angle of the longitudinal axis of the cylindrical lens in relation to the vertical or the horizontal.

With the known ophthalmic refractometers, the various refractive power parameters of the eye can be measured objectively. Therefore, each ophthalmic refractometer is provided with an optometer system for imaging a test mark on the retina of the eye and an observation system for observing the test mark imaged on the retina. A suitable beam splitter ensures separation of the incident beam entering the eye (optometer system) from the path of the beam emerging from the eye (observation system). The optometer system and the observation system may be adjusted in relation to a certain reference position in synchronization with one another with an appropriate change in their optical properties. The reference position here corresponds to the setting at which the test mark can be imaged by the optometer system in a good focus and can be observed with sharp contours by the observation system by using a test body whose the optical properties correspond to those of an ideal eye without a refractive power defect. Then if an eye with a refractive power defect is observed through the ophthalmic refractometer, the test mark in the reference position of the ophthalmic refractometer will appear blurred on the retina and will also be observed accordingly as a blurred image by the observation system.

Then, during the measurement, the optometer system and the observation system are adjusted in synchronization until the test mark is imaged on the retina in such a way that it is focused and can be observed with sharp contours by the observation system. Then a refractive power parameter can be determined from the difference between this setting and the reference position.

If the eye also has higher-order refractive power defects, the test mark will be imaged in various positions, the number of which corresponds to the ordinal number of the refractive power defect, each image appearing partially in sharp contours on the retina. On the basis of this finding, it is also possible to determine higher-order refractive power defects through a corresponding adjustment of the ophthalmic refractometer.

To measure the angle α, which indicates the position of the main axes in relation to the horizontal or vertical, for example, a so-called “Raubitschek curve” can be used as the test mark. Moving this Raubitschek curve into a position in which the test mark is focused on the retina for the first time allows the angle α to be determined.

U.S. Pat. No. 4,410,243 and German Patent DE 30 14 907 A1 disclose ophthalmic refractometers in which one or two eyepieces are provided in the observation system, so that the person performing the test can observe the test mark imaged on the retina through the eyepieces. The disadvantage of these systems is that it is relatively complicated for the person administering the test to measure the refractive power defect, and furthermore, the measurement accuracy depends on the care taken by the person administering the test.

German Patents DE 30 37 481 C2 and DE 31 02 450 C2 each describe ophthalmic refractometers in which devices for electronic measurement signal processing are provided in the observation system. Therefore, by appropriate analysis of the electronic measurement signals, it is possible to ascertain the settings of the ophthalmic refractometer in which the test mark is focused on the retina. One disadvantage of these devices is that they require complex instrument technology and often provide only an inadequate measurement quality.

Therefore, the object of the present invention is to propose a novel ophthalmic refractometer which avoids the disadvantages of the known state of the art. In addition, another object of the present invention is to provide a method of using an ophthalmic refractometer with which very good measurement results can be achieved easily.

This object is achieved by the ophthalmic refractometer and the method of using the ophthalmic refractometer according to the teaching of the two independent main claims.

Advantageous embodiments are the object of the subclaims.

According to this invention, a digital recording device is provided on the observation system of the ophthalmic refractometer and is connected to an image processing unit. The image processing unit may be designed in the manner of a standard computer, for example, in which suitable software is installed. As an alternative to that, implementation of image processing units in the form of hardware is also conceivable. According to the normal function of the ophthalmic refractometer, the test mark is imaged on the retina of the eye and is recorded by means of the digital recording device. The resulting digital image data of the test mark imaged on the retina is analyzed in the image processing unit, and one or more refractive power parameters of the eye are determined.

According to a preferred embodiment of this invention, the digital recording device is designed in the manner of a CCD chip. Such CCD chips have a sufficiently higher resolution and are available inexpensively.

Essentially it does not matter how the synchronized adjustment of the optometer system and the observation system is performed. A wide variety of possibilities for adjusting the optometer system and the observation system and for synchronizing the two adjusting movements are known from the state of the art. An especially simple design of the ophthalmic refractometer can be achieved if a linearly adjustable adjusting unit is provided on the ophthalmic refractometer, with a first and a second deflecting element being mounted thereon. The first deflecting element is arranged in the path of the beam of the optometer system, whereas the second deflecting element is arranged in the path of the beam of the observation system. The two deflecting elements are designed and arranged in such a way that the length of the path of the beam in the optometer system and in the observation system is varied by linear adjustment of the adjusting unit. Since both deflecting elements are fixedly connected to the adjusting unit in the same way, a synchronized change in length is implemented in the path of the beam of the optometer system and of the observation system by a simple linear adjustment of the adjusting unit.

A number of embodiments are conceivable for the design implementation of the two deflecting elements. According to a preferred embodiment, each deflecting element is formed by two reflection elements, e.g., mirrors or reflecting prisms, which are arranged in the path of the beam of the optometer system and/or of the observation system. The two reflective elements form a path like a trombone slide in the particular beam path, i.e., the beam of light is deflected 90° twice on the reflective elements, with the distance between the incident light striking the first reflective element and the beam of light reflected on the second reflective element remaining constant. Due to such a deflection of the light in the path of the beam of the optometer system and/or the observation system, it is readily possible to alter the length of the particular path of the beam by linear adjustment of the reflective elements arranged in pairs on the adjusting unit.

To be able to reduce the requirements regarding the manufacturing tolerances, it is especially advantageous if the deflecting elements are adjustably mounted on the adjusting unit. This makes it possible to accurately adjust the deflecting elements on the adjusting unit in assembly of the ophthalmic refractometer to permit accurate measurements. Even if measurement errors are found subsequently, the situation can be remedied by appropriate adjustment of the deflecting elements.

For accurate measurement of the refractive power of the eye, it is of crucial importance for the length of the path of the beam in the observation system to correspond exactly to the length of the path of the beam in the optometer system. To be able to implement this exact correspondence of the length of the two beam paths even when the beam path guides differ accordingly, there may be an offset in the adjustment direction between the first deflecting element and the second deflecting element. This offset is a fixedly predetermined offset, which makes it possible to compensate for differences in the length of other beam path segments.

Essentially it does not matter how the adjusting unit is driven for the adjustment. According to a preferred embodiment, however, the adjusting unit is driven by an electronic servomotor. The particular actual setting can be picked up on such a servomotor by using sensors, and this value can be sent to the analyzer unit as a corresponding adjustment value of the ophthalmic refractometer. In addition, the position of such a servomotor can be regulated so that the adjusting unit is able to approach precisely determined positions in which a digital image can then be made of the test mark imaged on the retina. The servomotor may then be operated like a stepping motor so that the adjustment of the control unit can be made in equidistant increments along the control path. Then a digital image is made at the equidistant stopping points of the adjusting unit and can be analyzed subsequently in the analyzer unit.

For example, the so-called Raubitschek curve can be used to determine the main axis angle α. However, this special test mark requires adjustment by an experienced operator, which is relatively time-consuming. Therefore, the use of a test mark is proposed, where this test mark is a contour image having midpoint symmetry with a plurality of contour transitions extending outward from the midpoint. The test mark here should preferably have a plurality of light and dark fields arranged in alternation. In particular, test marks that correspond in shape to a “Siemensstern” [Siemens star] are suitable for use with the ophthalmic refractometer according to this invention.

The inventive method of operating an ophthalmic refractometer is characterized in that first a plurality of digital image data records are recorded at different settings of the optometer system and/or the observation system by using the digital recording device and are stored together with the particular adjustment parameters associated with them. If a linearly adjustable adjusting unit is provided for adjustment of the optometer system and/or the observation system, then this adjusting unit is moved along the adjustment path, with one image of the test mark imaged on the retina being recorded at each of certain stopping points. Each of these digital photographs then forms an image data record and is stored together with the particular setting, i.e., with the corresponding linear manipulated variable in the present example.

According to this invention, the resulting image data records are analyzed by digital image processing in an image processing unit. According to a first embodiment, the image processing may take place in real time, so the image data records are analyzed in the image processing unit in parallel with being recorded by the digital recording unit. However, such real-time processing requires a very high computation power. If such computation power is not available, the image data records are first stored and then analyzed only after conclusion of recording all the image data records. This makes it possible for the photographs to be made very rapidly, so that the patient must position his eyes in front of the ophthalmic refractometer for only a relatively short period of time.

By using suitable computation methods accordingly, at least one contour sharpness evaluation is assigned to each image data record in the image processing unit. In other words, this means that the image of the test mark having sharp contours on the retina is evaluated in the image processing unit and is classified using one or more parameters.

If parameters for evaluating contour sharpness are available for all image data records, they are analyzed in an analyzer unit, e.g., by software installed on a standard computer. Then the analyzer unit can determine which settings of the ophthalmic refractometer yield relative maximum values for the contour sharpness evaluation. These relative maximums in the evaluation of the contour sharpness are characteristic of the refractive power of the eye, so that refractive power parameters of the eye being tested can be derived by the analyzer unit from the difference between the settings in which relative maximum values for the contour sharpness evaluation are obtained.

In optometry, usually only first- and second-order refractive power defects are taken into account. It is therefore especially advantageous if the image data records having the two highest relative maximum values can be determined in the analyzer unit. The refractive power parameters in the two main axes of the refractive power can be derived from the settings of the ophthalmic refractometer assigned to these relative maximum values, so that the usual parameters can be converted quickly for production of the spherocylindrical lens required for correction of the refractive power defect.

If the angle α is also determined in addition to the two diopter values for the refractive power correction in the two main axes, then an advantageous variant of this method may be used. In this variant of the method, the test mark used is a contour image having midpoint symmetry and a plurality of contour transitions extending outward from a midpoint. If the eye to be examined has a refractive power defect of the second order, this has the result that this contour image having midpoint symmetry is not imaged in completely good focus at any setting of the ophthalmic refractometer. Instead, the image data records with the relative maximum values for the contour sharpness evaluation have sharp contours only in some areas. Other areas, however, are imaged only with a slight blur even in these image data records having relative maximum values for the contour sharpness evaluation. To derive the angle α for the contour sharpness evaluation, the particular contour transition at which the test mark is imaged with maximum contour sharpness is sought among the image data records having relative maximum values. The angle α is then derived from the angle difference between this contour transition having maximum contour sharpness and the vertical or horizontal line.

Different computation algorithms may be used for the contour sharpness evaluation of the image data records required for performing this method.

If fixation lighting is provided on the ophthalmic refractometer for fixation of the eye in a position in which the fundus of the eye, in particular the nerve fiber head, can be observed with the observation system, then the ophthalmic refractometer can also be used as a fundus camera without any major additional measures. For unstructured illumination of the fundus, it is necessary only to provide a suitable lighting device accordingly. By appropriate analysis of the image data, it is also possible in particular to determine the depth of excavitation of the nerve fiber head.

This invention is explained in greater detail below on the basis of the drawings as an example.

They show:

FIG. 1 an emmetropic eye (having normal vision) in a schematic cross section;

FIG. 2 a myopic eye (nearsighted) in a schematic cross section;

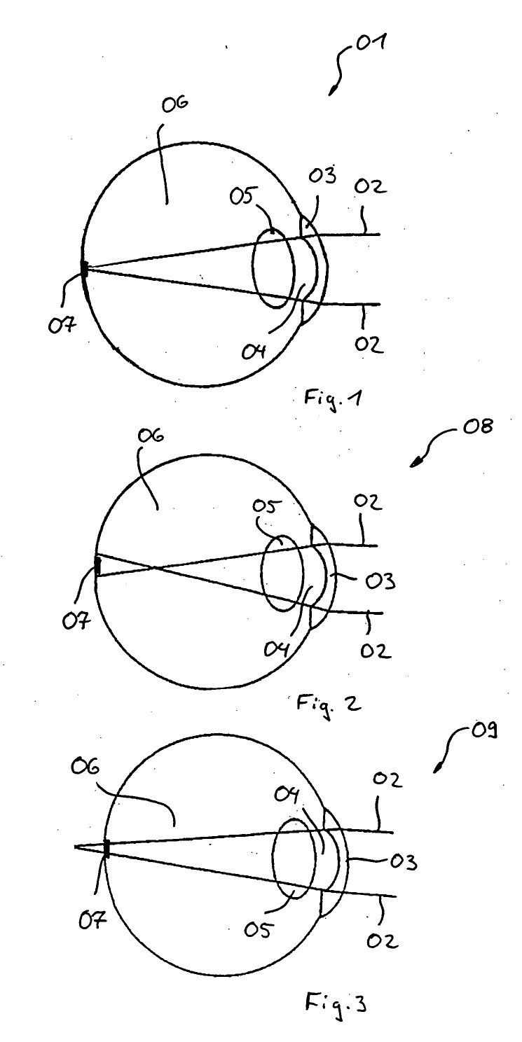

FIG. 3 a hypermetropic eye (farsighted) in a schematic cross section;

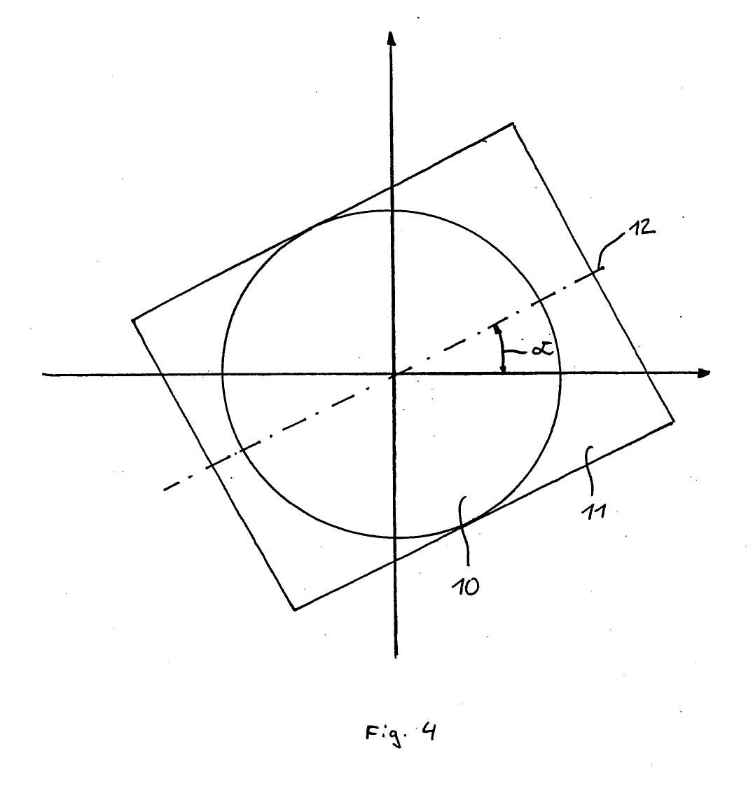

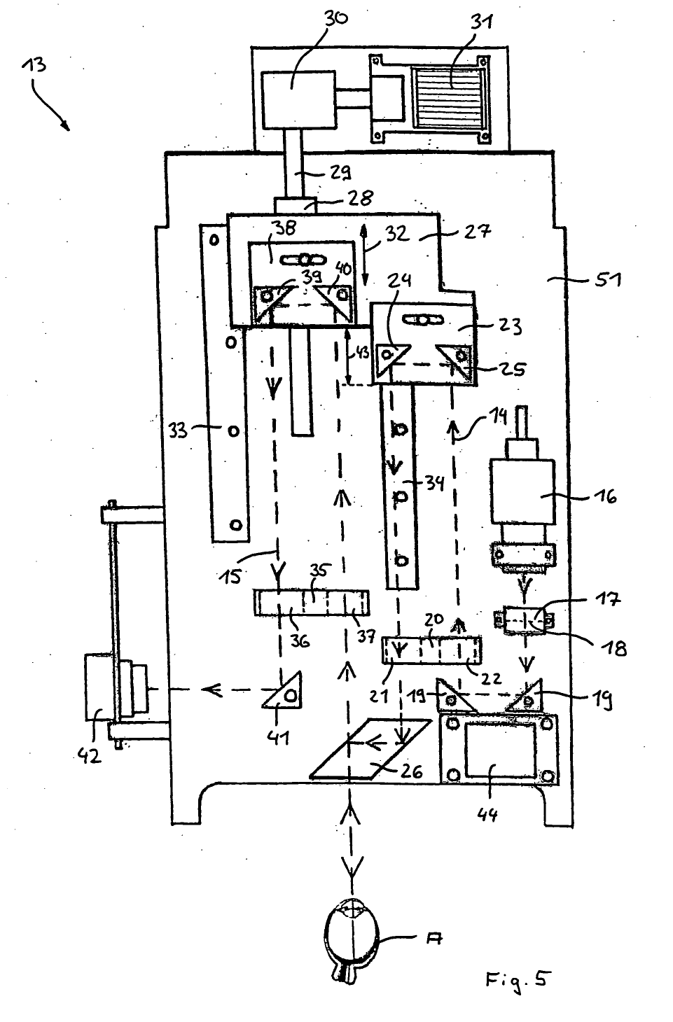

FIG. 4 a spherocylindrical corrective lens for correcting refractive power defects of the first and second order in a schematic view as seen from the front;

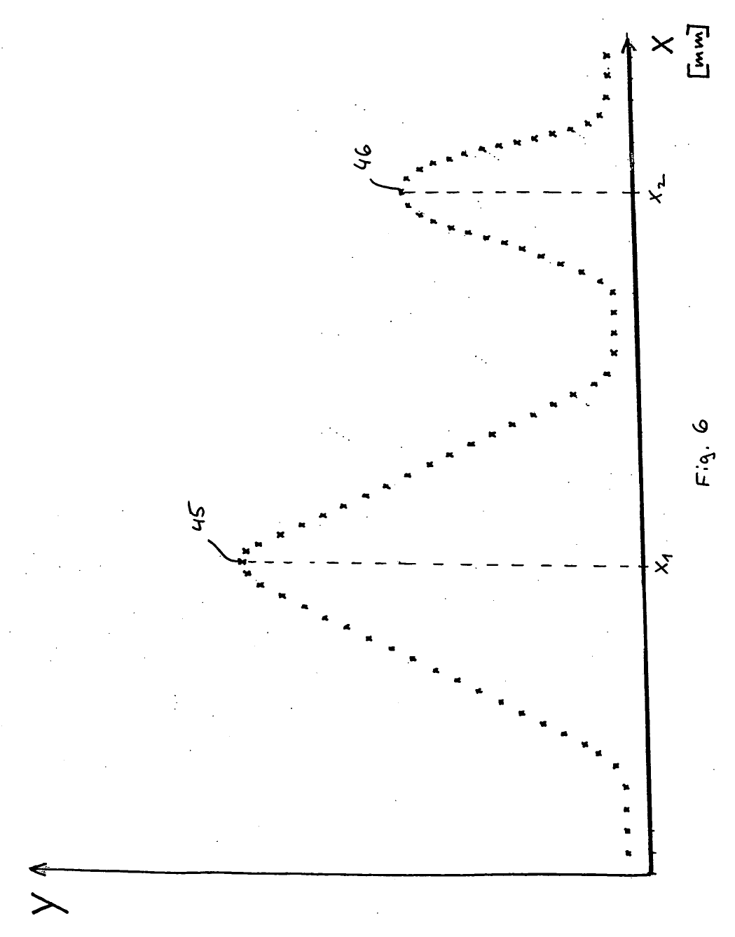

FIG. 5 an ophthalmic refractometer in a schematic view as seen from the front;

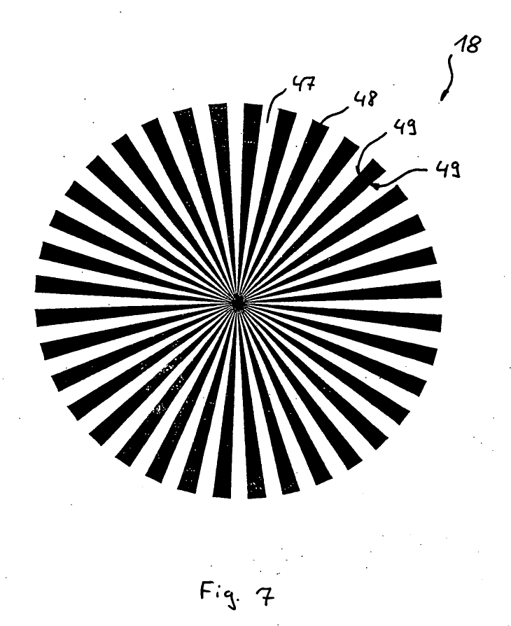

FIG. 6 a schematic measurement diagram of the contour sharpness evaluation of various photographs of the test mark, plotted over the adjustment path of the ophthalmic refractometer;

FIG. 7 the contour image of a test mark suitable for being imaged on the retina of the eye;

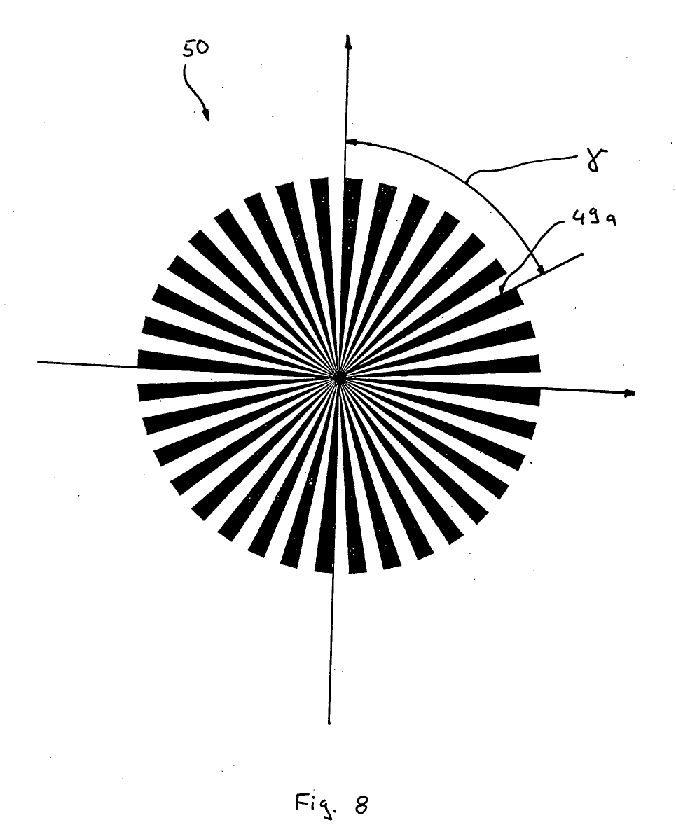

FIG. 8 a schematic diagram of the image of the test mark on the retina as shown in FIG. 7.

FIG. 1 shows the condition of an eye 01 with normal vision, shown schematically in cross section. As shown in FIG. 1, the parallel rays of light 02 coming from the environment enter the eye 01 through the cornea 03, pass through the cornea 03, the aqueous humor 04, the lens 05 and the vitreous humor 06. The light rays 02 are focused by the refractive power of the cornea 03, the aqueous humor 04, the lens 05 and the vitreous humor 06, focusing them on a focal point, which is located on the retina 07 of the eye 01 in the case of normal vision as depicted in FIG. 1.

However, in the case of a myopic eye 08, as depicted in FIG. 2, the incident rays of light 02 arriving in parallel from the environment do not strike the retina 07, but instead are focused just in front of it, so that only a blurred image is obtained on the retina 07.

However, if an eye 09 is hypermetropic, as depicted in FIG. 3, then the parallel incident rays of light 02 coming from the environment strike the retina 07 just behind the retina 07, which in turn also results in blurred vision.

The vision defects depicted in FIG. 2 and FIG. 3 are referred to as first-order refractive power defects and can be corrected by wearing eyeglass lenses with a spherical correction. The corresponding spherical eyeglass lens must be designed so that the incident rays of light from the environment strike the retina 07.

In addition to the first-order refractive power defects depicted in FIG. 2 and FIG. 3, second-order refractive power defects may also occur in the eye. Such second-order refractive power defects mean that the refractive power of the eye is not the same at all points but instead depends on the angle in relation to the vertical or horizontal. Such second-order refractive power defects can be corrected by using a cylindrical corrective lens. Therefore, with the usual visual aids such as eyeglasses or contact lenses, three refractive power parameters are given so that these lenses can be produced. The first refractive power parameter D1 indicates the refractive power of the spherical corrective lens. The second refractive power parameter D2 indicates the refractive power of the cylindrical corrective lens. The refractive power is given here in diopters. The third refractive power parameter indicates the angle α at which the center axis of the cylindrical corrective lens extends in relation to the horizontal or vertical.

FIG. 4 shows schematically a view of a spherocylindrical corrective lens as seen from the front. This shows that the corrective lens consists of a combination of a spherical corrective lens 10 and a cylindrical corrective lens 11 either in front of or behind the former. The corrective lens shown in FIG. 4 must be described by stating the refractive power of the spherical corrective lens 10, the refractive power of the cylindrical corrective lens 11 and the angle α between the center axis 12 of the cylindrical corrective lens and the horizontal (or vertical).

FIG. 5 shows an inventive ophthalmic refractometer 13 in a schematic view from above. This ophthalmic refractometer 13 has an optometer system with which a beam of light 14 can be directed at the retina of an eye A to be examined. In addition, the ophthalmic refractometer 13 also has an observation system, so that the retina of the eye to be examined can be observed via the beam 15.

The optometer system of the ophthalmic refractometer 13 comprises essentially a light source 16 (which may be designed in the manner of a light-emitting device or LED, for example), a test mark holder 17 with a test mark 18 mounted therein (see FIG. 7), two reflective elements 19 in a stationary mount, a carrier element 20 with two lenses 21 and 22 mounted therein, a first deflecting element 23 with two reflective elements 24 and 25 mounted thereon and a reflecting prism 26. The light source 16, the test mark holder 17, the reflective elements 19, the carrier element 20 with the lenses 21 and 22 and the reflecting prism 26 are mounted in a stationary position on a base plate 51. However, the deflecting element 23 with the reflective elements 24 and 25 is adjustably mounted on an adjusting unit 27 designed in the manner of a carriage. The adjusting unit 27 engages with a servomotor 31 via a spindle nut 28, a drive shaft 29 and a gear 30, so that by driving the servomotor 31, the adjusting unit 27 can be adjusted in the direction of the arrow 32 indicating movement. To do so the adjusting unit 27 is mounted so it is linearly adjustable on two rails 33 and 34, which are adjustably mounted on the base plate 51.

As can be seen from the beam 14 of the optometer system, the light emitted by the light source 16 passes through the test mark holder 17, resulting in a contour image of the test mark 18 provided there. By reflection on the reflective elements 19, 24, 25 and on the reflective surfaces of the reflecting prism 26, the beam of light is deflected onto the retina of the eye A so that the contour image of the test mark 18 is imaged there. The length of the beam 14 can be lengthened or shortened by adjusting the adjusting unit 27. The arrangement of the reflective elements 24 and 25 of the first deflecting element 23 in relation to the left reflective element 19 and the reflecting prism 26 form a path resembling a trombone slide. An adjustment of the adjusting unit 27 by one unit of measure therefore results in the beam 14 being shortened or lengthened by exactly two units of measure in each case.

The observation system of the ophthalmic refractometer 13 is formed by a carrier element 35 with two lenses 36 and 37 mounted in it, a second deflecting element 38 having two reflective elements 39 and 40 attached to it, a reflective element 41 and a digital recording device 42 designed in the manner of a CCD chip.

As can be seen from the beam path 15 of the observation system, the contour image which is imaged on the retina of the eye A is reflected on the retina and can be observed by reflection on the reflective elements 39, 40 and 41 by means of the camera device 42. The carrier element 35 with the lenses 36 and 37 and the reflective element 41 are in turn mounted in a stationary mount on the base plate 51. Like the first deflecting element 23, the second deflecting element 38 with the reflective elements 39 and 41 is mounted adjustably on the adjusting unit 27 and can therefore be adjusted by the drive of the servomotor 31 in synchronization with the first deflecting element 23. The two deflecting elements 23 and 38 are mounted jointly on the adjusting element 27 and form a section of a beam path resembling a trombone slide, so this greatly simplifies synchronization of the adjustment of the optometer system and the observation system because there is no mechanical coupling of the two systems for synchronized transmission of the adjusting movement. Since beam path 14 and beam path 15 must be of exactly the same length, the deflecting elements 23 and 38 are mounted on the adjusting unit 27 with an offset 43. This offset 43 compensates for differences in running length of the beam paths 14 and 15 resulting from the difference in arrangement of the different reflective elements.

The beam paths 14 and 15 each pass through two lenses 21 and 22 and/or 36 and 37, so an adjustment of the adjusting unit 27 and the associated shortening or lengthening of the beam paths 14 and 15 result in a change in focus in the optometer system and the observation system. These adjustments in focus are preferably performed in synchronization because due to the joint arrangement of the deflecting elements 23 and 38 on the adjusting unit 27, the change in running length in the beam paths 14 and 15 is exactly the same in each case.

Before using the ophthalmic refractometer 13, it must first be calibrated. To do so, instead of the eye A, a test body whose optical properties correspond to those of an ideal eye without any refractive power defect is used. The adjusting unit 27 is then adjusted along the adjustment path until a sharply contoured image of the test mark 18 is recorded by the recording device 42. The corresponding position of the adjusting unit 27 is stored as the reference position or the zero position.

To measure the refractive force parameter of an eye A, the following procedure is then used:

First, adjusting unit 27 is moved by the drive of the servomotor 31 to the rear border of the adjusting region, which corresponds to the position depicted in FIG. 5. Then the eye A is aligned by the person performing the test in relation to the base plate 51 of ophthalmic refractometer 13. Therefore, the person performing the test uses a device 44, which permits direct observation of the eye A by deflection on the reflecting prism 26. Then by means of the device 44, a fixation mark is faded into the eye A from above via the reflecting prism 26 to fix the pupil of the eye in a predetermined axis.

After the eye A has been aligned optimally in this way, the light source 16 is activated to image the test mark 18 on the retina of the eye A via the beam path 14. This image on the retina is observed by the recording device 42 via the beam path 15, so that a digital image is prepared by the recording device 42 and can be stored as a first image data record. Then the adjusting unit 27 is moved forward by a certain amount in the direction of the arrow 32 and is stopped again a certain distance from the starting position. With this new focusing of beam paths 14 and 15, a digital image is again recorded by the recording device 42 and stored as an additional image data record. In this way the adjusting unit is then moved forward in a plurality of equidistant increments, with a digital image of the test mark 18 imaged on the retina being recorded and stored by the recording device 42 at each individual stopping point. In addition, in storage of the image data records, the position of the adjusting unit 27 and/or the servomotor 31 in relation to the reference position or zero position is also added to each image data record and is stored with the proper assignment. As soon as the adjusting unit 27 has reached the forward end of the adjusting region, then the person performing the test can remove the eye A from the fixed position. After conclusion of this recording cycle, a data record is available, consisting of a plurality of digital images of the test mark 17 imaged on the retina, with a certain position of the adjusting unit 27 in relation to the reference position or zero position being assigned to each image recorded.

Then in the next step, the individual image data records are analyzed by an image processing unit, which may be implemented, for example, by installation of suitable software on a standard computer. Through appropriate analytical algorithms, at least one value Y is assigned to each image data record, where Y classifies the sharpness of the contour of the image represented by the image data record.

FIG. 6 shows a schematic diagram in which the contour sharpness evaluation Y of the various image data records in one measurement cycle is plotted as a function of the adjusting region of the adjusting unit 27. It can be seen here that the values for the contour sharpness evaluation assume two relative maximum values 45 and 46 in the adjusting area of the adjusting unit 27. The two refractive power parameters D1 and D2 which determine the refractive power of the spherocylindrical contour lens, can be derived from the assigned manipulated variables X1 and X2 of adjusting unit 27 in relation to the reference position determined in calibration.

To also be able to derive the angle α from the measurements using the ophthalmic refractometer 13, the test mark shown in FIG. 8 is used. The test mark 18 is designed in the manner of a Siemens star having a plurality of light and dark fields 47 and 48 arranged in alternation. The light and dark fields 47 and 48 are designed in the form of segments of a circle, thus resulting in a contour with midpoint symmetry and with contour transitions 49 that extend outward.

The digital image 50 shown in FIG. 8 is an image of the test mark 18 recorded at position X2, i.e., at the lower relative maximum value 46. The diagram in FIG. 8 is merely an example, because due to the second-order refractive power defects of the eye A, only partial regions of the test mark 18 are imaged with sharp contours in an actual photograph, whereas other areas appear slightly blurred.

Through suitable image processing algorithms, the contour transition 49a in the digital image 50 having the highest relative contour sharpness in the image 50 is determined. Then by a suitable mathematical conversion, a refractive power parameter, namely the angle α, can be derived from the angle γ between the vertical or horizontal.

List of Reference Notation

| | |
|---|---|
| 01 | eye (normal vision) |
| 02 | ray of light |
| 03 | cornea |
| 04 | aqueous humor |
| 05 | lens |
| 06 | vitreous humor |
| 07 | retina |
| 08 | eye (myopic) |
| 09 | eye (hypermetropic) |
| 10 | spherical corrective |
| | lens |
| 11 | cylindrical corrective |
| | lens |
| 12 | center axis (cylindrical |
| | corrective lens) |
| 13 | refractometer |
| 14 | beam path, optometer |
| | system |
| 15 | beam path, |
| | observation system |
| 16 | light source (LED) |
| 17 | test mark holder |
| 18 | test mark |
| 19 | reflective element |
| 20 | carrier element |
| 21 | lens |
| 22 | lens |
| 23 | first deflecting element |
| 24 | reflector element |
| 25 | reflector element |
| 26 | reflecting prism |
| 27 | adjusting unit |
| 28 | shaft nut |
| 29 | drive shaft |
| 30 | gear |
| 31 | servomotor |
| 32 | direction of adjustment |
| 34 | rail |
| 35 | carrier element |
| 36 | lens |
| 37 | lens |
| 38 | second deflecting element |
| 39 | reflector elements |
| 40 | reflector elements |
| 41 | reflector elements |
| 42 | digital recording device |
| 43 | offset between first and |
| | second deflecting elements |
| 44 | device for blending in a |
| | fixation mark and for |
| | direct observation of the |
| | eye |
| 45 | first relative maximum |
| | value of the contour |
| | sharpness evaluation Y |
| 46 | second relative maximum |
| | value of the contour |
| | sharpness evaluation Y |
| 47 | brightness field of the |
| | test mark |
| 48 | darkness field of the test |
| | mark |
| 49 | contour transition |
| 50 | digital image recording |
| 51 | base plate |
| A | eye |

1. An ophthalmic refractometer (13) for objective determination of the refractive power of an eye (A), comprising an optometer system for imaging a test mark on the retina of the eye (A) and comprising an observation system (15) for observation of the test mark (18) imaged on the retina, whereby the optometer system and the observation system can be adjusted in mutual synchronization in relation to a reference position, and whereby a refractive power parameter of the eye (A) can be determined from the difference between the reference setting and the setting at which the test mark (18) is imaged on the retina with at least some sharp contours,
characterized in that a digital recording device and a digital processing unit are provided on the observation system, whereby digital image data of the test mark (18) imaged on the retina can be recorded with the recording device and the image data thus obtained can be analyzed in the image processing unit by digital image processing to determine the refractive power parameter.

2. The ophthalmic refractometer according to claim 1, characterized in that the digital recording device is designed in the manner of a CCD chip.

3. The ophthalmic refractometer according to claim 1 or 2, characterized in that a linearly adjustable adjusting unit (27) is provided for synchronous adjustment of the optometer system and the observation system, a first deflecting element (23) which is situated in the beam path (14) of the optometer system and a second deflector element (38) which is situated in the beam path (15) of the observation system being mounted thereon, so that by adjusting the adjusting unit (27), the length of the beam path (14) in the optometer system and the length of the beam path (15) in the observation system are varied in mutual synchronization.

4. The ophthalmic refractometer according to claim 3, characterized in that the first deflecting element
and/or the second deflecting element (38) each have two reflective elements (24, 25; 39, 40) which are arranged in the beam path (14) of the optometer system and/or in the beam path (15) of the observation system, so that a path is formed resembling a trombone slide, suitable for varying the length of the beam path (14) in the optometer system and/or the length of the beam path (15) in the observation system.

5. The ophthalmic refractometer system according to claim 3 or 4, characterized in that the first deflecting element (23) and/or the second deflecting element (38) are adjustably mounted on the adjusting element (27).

6. The ophthalmic refractometer according to one of claims 3 through 5, characterized in that the first deflecting element (23) and/or the second deflecting element (38) are mounted on the adjusting unit (27) with a mutual offset (43), so the length of the beam path (14) in the optometer system between the test mark (18) and the eye (A) corresponds exactly to the length of the beam path (15) in the observation system between the eye (A) and the digital recording unit.

7. The ophthalmic refractometer according to one of claims 3 through 6, characterized in that the adjusting unit (27) can be adjusted using a servomotor (31).

8. The ophthalmic refractometer according to one of claims 3 through 7, characterized in that the position of the adjusting unit (27) can be detected with a sensor, whereby a zero position of the adjusting unit (27) defines the reference position of the ophthalmic refractometer (13), and the position of the adjusting unit (27) measured by the sensor is relayed to an analyzer unit, where it can be analyzed as a current setting of the ophthalmic refractometer (13) in relation to the reference position.

9. The ophthalmic refractometer according to claim 8, characterized in that the position of the adjusting unit (27) can be detected by analysis of the position of the servomotor (31).

10. The ophthalmic refractometer according to one of claims 7 through 9, characterized in that the servomotor (31) can be operated as a stepping motor for adjusting the adjusting unit (27) in equidistant increments.

11. The ophthalmic refractometer according to one of claims 1 through 10, characterized in that a contour image having midpoint symmetry, where a plurality of contour transitions (49) extend outward from the midpoint, is used as the test mark (18).

12. The ophthalmic refractometer according to one of claims 1 through 11, characterized in that the test mark (18) has a plurality of light fields (47) and dark fields (48) arranged in alternation.

13. The ophthalmic refractometer according to one of claims 1 through 12, characterized in that an LED lighting device (16) for illuminating the test mark (18) is provided on the optometer system.

14. The ophthalmic refractometer according to one of claims 1 through 13, characterized in that at least two lenses (21, 22; 36, 37) arranged in a stationary mount are provided in the beam path (14) of the optometer system and/or in the beam path (15) of the observation system.

15. The ophthalmic refractometer according to claim 14, characterized in that the lenses (21, 22; 36, 37) are mounted on a common carrier element (20, 35) so that the lenses (21, 22; 36, 37) are arranged in the area of the part of the beam (14, 15) resembling a trombone slide.

16. The ophthalmic refractometer according to one of claims 1 through 15, characterized in that a device (44) for applying a fixation mark to the eye (A) is provided.

17. The ophthalmic refractometer according to one of claims 1 through 16, characterized in that a device (44) for direct observation of the eye (A) by a person performing the test is provided.

18. The ophthalmic refractometer according to one of claims 1 through 17, characterized in that a fixation light is provided for fixation of the eye in a position in which the fundus of the eye, in particular the nerve fiber head, can be recorded with the observation system.

19. A method of operating an ophthalmic refractometer (13) for objective determination of the refractive power of an eye (A) comprising an optometer system for imaging a test mark (18) on the retina of the eye (A) and comprising an observation system for observation of the test mark (18) imaged on the retina of the eye (A), whereby the optometer system and the observation system can be adjusted in mutual synchronization and in relation to a reference position and whereby a refractive power parameter of the eye (A) can be determined from the difference between the reference position and the position at which the test mark (18) is imaged on the retina with at least partially sharp contours, characterized in that
at different settings of the optometer system and the observation system, multiple digital image data records are recorded using a digital recording device (44) and are stored together with the particular assigned adjustment parameters of the optometer system and the observation system, whereby each individual image data record represents the test mark (18) imaged on the retina at the particular setting;
the image data records are analyzed by digital processing in an image processing unit to assign a contour sharpness evaluation to each image data record;
the contour sharpness evaluations of the image data records are analyzed in an analyzer unit to ascertain the settings at which relative maximum values (45, 46) for the contour sharpness evaluation are obtained;
a refractive power parameter is determined in the analyzer unit from the difference between the reference setting and the settings at which there are relative maximum values (45, 46) for the contour sharpness evaluation.

20. The method according to claim 19, characterized in that the image data records having the two highest relative maximum values (45, 46) for the contour sharpness evaluation are determined in the analyzer unit, and the refractive power parameters in the two main refractive power axes are derived from the corresponding assigned settings.

21. The method according to claim 19 or 20, characterized in that a contour image having a plurality of contour transitions (49) extending outward from a midpoint is used as the test mark (18), and an image data record having a particularly low maximum value (46) for the contour sharpness evaluation is analyzed in the image processing unit, said contour transition (49a) of the test mark (18) having the maximum contour sharpness is imaged and the angle of inclination (α) of the main refractive power axes with respect to the horizontal and/or vertical is derived from the angular difference (γ) between this contour transition (49a) and the vertical or horizontal respectively.

22. The method according to claim 19, characterized in that
by means of a fixation light, the eye is fixed in a position in which the nerve fiber head can be observed by the observation system;
multiple digital image data records are recorded at different settings of the observation system using the digital recording device and these image data records are stored together with the setting parameters of the observation correlated with them, whereby each individual image data record represents the nerve fiber head as observed in the particular setting;
in an image processing unit the image data records are analyzed by digital image processing so that a contour sharpness evaluation can be assigned to each image data record;
in an analyzer unit the contour sharpness evaluations of the image data records are analyzed to ascertain the settings at which relative maximum values for the contour sharpness evaluation are obtained;
the depth of the excavitation of the nerve fiber head is determined in the analyzer unit from the difference between the settings at which relative maximum values for the contour sharpness evaluation are obtained.
